(19) United States
(12) Patent Application Publication
(10) Pub. No.: US 2004/0223891 A1
(43) Pub. Date: Nov. 11, 2004

(54) Fragrance dispensing air register vent

(76) Inventor: Barry L. Brown, Indianapolis, IN (US)

Correspondence Address: BOWERS HARRISON LLP, GARY K. PRICE, ESP., 25 RIVERSIDE DRIVE, PO BOX 1287, EVANSVILLE, IN, 47706-1287, US

(21) Appl. No.: 10/841,015

(22) Filed: May 5, 2004

Related U.S. Application Data
(60) Provisional application No. 60/467,459, filed on May 5, 2003.

(51) Int. Cl. A61L 9/12

(52) U.S. Cl. 422/124

(57) ABSTRACT

A fragrance dispensing air register vent of a central heat or air conditioner system or to a main return vent. The air register vent includes a door mounted to a top surface of the register vent to give access to a fragrance package. As such, to replace the scent package, the vent does not first require being physically removed from the floor, wall or ceiling. In application the door is hingedly attached to the upper surface so as to enable the door to be pivotably opened for access to the fragrance package, and closed. The vent register further includes a fragrance holder disposed immediately below the door in order to have access to the contents of the holder. The holder including a bottom surface having a plurality of apertures, the apertures for allowing air from the distribution duct to freely pass through the bottom surface of the fragrance holder. The door likewise includes a plurality of openings for allowing air that passes from the distribution duct through the fragrance holder to freely pass through the door of the upper surface. Since the fragrance holder is provided with the apertures and the door of the vent is provided with the plurality of openings, the air from the distribution duct will have little resistance from the air register vent and will pass into the room with a minimum of resistance from the air register vent.

### CROSS REFERENCES TO RELATED APPLICATIONS

U.S. Provisional Application for Patent 60/467,459, filed May 5, 2003, with title, “Fragrance Dispensing Air Register Vent” which is hereby incorporated by reference. Applicant claims priority pursuant to 35 U.S.C. par. 119(e)(i).

### STATEMENT AS TO RIGHTS TO INVENTIONS MADE UNDER FEDERALLY SPONSORED RESEARCH AND DEVELOPMENT

Not Applicable

### BACKGROUND OF THE INVENTION

1. Field of the Invention

The present invention relates to the field of home and business ventilation systems, and more particularly to a room air freshener to be used in connection with a register vent.

2. Brief Description of Prior Art.

This invention pertains to a fragrance dispensing air register vent such as may be used in rooms required to be central heated or cooled for living or working. Prior art devices have been used for dispensing aromatic scents in a room of a house or business. Furthermore, different scents have been added to a furnace or air conditioner filter such that the air flow from the furnace or air conditioner will blow through the filter and as such, force the scent from the filter out through the different outlet vents of different rooms provided for the furnace or air conditioner. Different types of filters and materials have been used for such purposes. However, locating and opening the furnace can be a dirty job and a disadvantage to this type system. Further, with this type system the same fragrance is circulated throughout the entire home or business.

Scent packages having different fragrances are also available and can be used in local areas of a home or business for applying different scents in different areas of the building. However there is no way to circulate such fragrances. Some scent devices have made use of an electrical heating or blowing device which requires the use of an available electrical plug which is not always readily available in the area in which one desires to add fragrance.

There also exists prior art outlet vents of a central heat or air conditioner system, such vents adapted to receive a scent package. However, in order to access the scent package of these prior art vent registers, the vent registers must first be physically removed from the floor, wall or ceiling. This procedure is not only inconvenient for the user, but can also turn into a rather dusty and dirty job.

As will be seen from the subsequent description, the preferred embodiments of the present invention overcome shortcomings of the prior art.

### SUMMARY OF THE INVENTION

The present invention overcomes the adversities of the prior art and provides a simple fragrance dispensing air register vent of a central heat or air conditioner system or to a main return vent. In this regard, different fragrance packages can be directed into each different room. Thus, any desired fragrance can be added to each room. The air register vent of the present invention includes a hinged door disposed on a top surface of the register vent to give access to a fragrance package. As such, to replace the scent package, the vent does not first require being physically removed from the floor, wall or ceiling.

The invention will be better understood and further objects and advantages thereof will become more apparent from the ensuing detailed description of a preferred embodiment taken in conjunction with the drawings.

### BRIEF DESCRIPTION OF THE DRAWINGS

FIG. 1 is a perspective view of the preferred embodiment of the present invention, a fragrance dispensing air register vent, with the hinged door in an open position.

FIG. 2 is a fragrance holder of the air register vent of FIG. 1.

FIG. 3 is a perspective view of the air register vent of FIG. 1, with the hinged door in a closed position.

### DESCRIPTION OF THE PREFERRED EMBODIMENT

In accordance with the present invention, a fragrance dispensing air register is disclosed. The device providing a fragrance dispensing air register vent of a central heat or air conditioner system or to a main return vent. Specifically, it will be noted in the drawings that the fragrance dispensing air register vent provides a means for individually replacing the fragrance package from the fragrance dispensing air register vent without the user being required to first physically remove the air register vent from the floor, wall or ceiling. In the broadest context, the fragrance dispensing air register vent consists of components configured and collated with respect to each other so as to attain the desired objective.

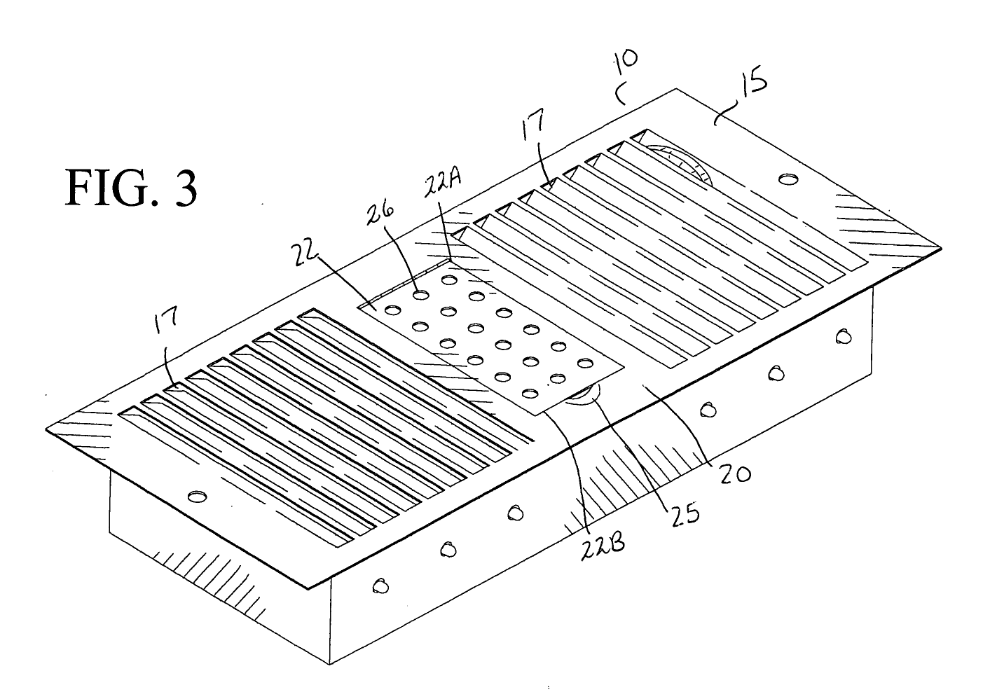

Referring to FIGS. 1-3, a fragrance dispensing air register vent 10 is disclosed. The vent 10 having a typical configuration of an air register as it exists in the prior art. In general, the vent 10 is placed in fluid communication with the distal end of an air distribution duct (not shown) and is mounted with an upper surface 15 parallel to and in contact with a floor, wall, or ceiling within the room being served.

Upper surface 15 of air.register vent 10 having a series of parallel air outlet flow vanes 17, the air outlet flow vanes 17 define air passage ways and which are divided into two sets 17A and 17B. Sets 17A, B of flow vanes 17 differ in that they are directed away from each other, each set directing air flow toward the left and right ends of the upper surface 15, respectively. In this manner air flowing through air register vent 10 is dispersed as it enters the room and is distributed broadly.

Disposed between sets 17A,B of flow vanes 17 is a bridge 20 having a chamber 21 therein. The chamber 21 is defined by interior walls 21A that downwardly extend from the upper surface 15 of the vent 10. The walls 21A define an opening 21B disposed in the bridge 20 of the upper surface 15. The walls 21A further include a projecting shelf 21C in perpendicular relationship to the wall 21A. The shelf 21C extends the perimeter of the walls 21A of the chamber 21.

A door 22 is hingedly attached to the bridge 20 and above the opening 21B of the chamber 21. As shown in FIG. 1, the door 22 includes a first end 22A that is hinged to the top surface 15, and a second end 22B opposite the first end 22A.

The top surface 15 further including an indented portion 25 disposed on the bridge 20 and adjacent to the second end 22B of the door 22. The door 22 is hingedly opened in an upward direction as shown in FIG. 1 by lifting the second end 22B of the door. As should be understood, the indented portion 25 allows the user sufficient contact with the second end 22B in order to hingedly open the door 22 as described above. As shown in the drawings, the length of the door 22 is approximately parallel with the length of each of the flow vanes 17.

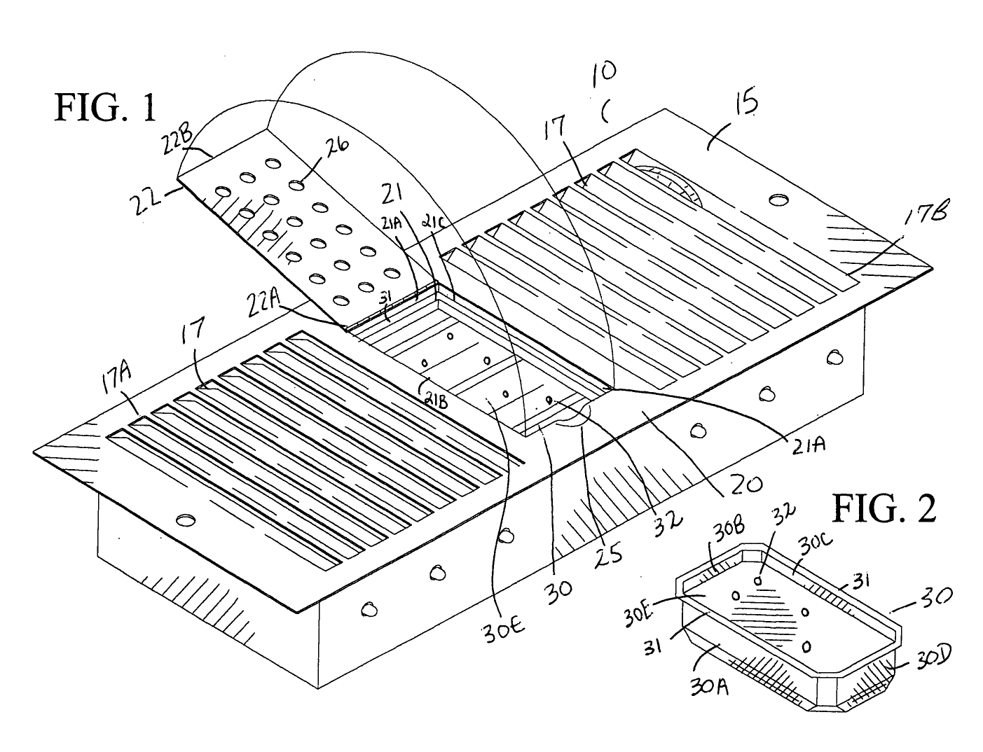

A fragrance holder 30 (best shown in FIG. 2) is removably disposed within the chamber 21 so that access to the contents of the holder 30 is through the opening 21B by lifting the door 22 as discussed above. The holder 30 having four sides 30A,B,C,D, and a bottom surface 30E. The top of the holder 30, opposite the bottom surface 30E remains open.

The sides 30A, B, C, D of the holder 30 define a rim 31. In application, the holder 30 is positioned within the chamber 21 so that the rim 31 rests on the shelf 21C disposed within the chamber 21.

The bottom surface 30E of the holder 30 further includes a plurality of apertures 32 therethrough, the apertures 32 for allowing air from the distribution duct to freely pass through the bottom surface 30E of the fragrance holder 30. The number and size of the apertures 32 may be varied so as to provide for the optimum usefulness.

It is necessary that the fragrance holder 30 and particularly the sides of 30A, 30C of the fragrance holder 30 avoids interference with the flow vanes 17. This is done simply by sizing the fragrance holder 30 to that of the chamber 21.

The door 22 further includes a plurality of bores 26 therethrough, the bores 26 for allowing air that passes from the distribution duct through the fragrance holder 30 to freely pass through the door 22. Since the fragrance holder 30 is provided with the apertures 32 and the door 22 of the vent 10 is provided with the plurality of bores 26, the air from the distribution duct will have little resistance from the air register vent 10 and will pass into the room with a minimum of resistance from the air register vent 10.

Fragrance packages (not shown) of different sizes and configurations, and having different fragrances are available on the market. In application, the fragrance package is placed within the fragrance holder 30 by lifting the door 22 as discussed above. The fragrance package lies in the holder 30 so that air flow will go through the apertures 32 to pick up the scent impregnated in the fragrance package. The scented air then passes through the openings 26 in the door 22, and then into the room.

The fragrance material is a volatile substance, preferably in a solid form, such as pellets of fragrance, or a single solid bar. However other forms of odor improving chemical may occur to those skilled in the art.

The hinged door 22 provides a simple way to insert and/or replace the fragrance package within the fragrance holder 30 without the user being required to first physically remove the air register vent from the floor, wall or ceiling. Thus, the invention provides a fragrance dispensing air register vent that is convenient to use, inexpensive to manufacture, and adapted to avoid the inconvenience and rather dusty and dirty job of replacing scent packages in prior art vent registers.

Although the description above contains many specificities, these should not be construed as limiting the scope of the invention but as merely providing illustrations of some of the presently preferred embodiments of the invention. For example, while the fragrance holder 30 has been described as being removably positioned within the chamber 21, it should be understood that the holder 30 can be permanently affixed within the chamber 21, or manufactures integral with the vent 10.

This invention thus described, it will be obvious that the same may be varied in many ways. Such variations are not to be regarded as a departure from the spirit and scope of the invention, and all such modifications as would be obvious to one skilled in the art are intended to be included within the scope of the present invention.

Thus the scope of the invention should be determined by the appended claims in the formal application and their legal equivalents, rather than by the examples given.

I claim:

1. A fragrance dispensing air register vent comprising:
an upper surface having a plurality of air flow vanes positioned to define air passages and direct the flow of air passing therethrough, said upper surface further including an opening,
a chamber disposed within the vent, said chamber positioned adjacent the opening,
a door hingedly attached to the upper surface such that the door is positioned over the opening, said door having a plurality of bores capable of passing air therethrough,
a fragrance holder disposed within the chamber, said holder adapted to contain a fragrance material and capable of dispensing a fragrance therefrom, said holder including a bottom surface having a plurality of apertures capable of passing air therethrough.

2. The air register vent as recited in claim 1, wherein the chamber includes a projecting shelf.

3. The air register vent as recited in claim 2, wherein the fragrance holder further includes four sides that define a rim, wherein the fragrance holder is positioned within the chamber such that the rim rests on the shelf of the chamber.

4. The air vent register as recited in claim 1, wherein the fragrance holder is aligned beneath the door.

5. The air register vent as recited in claim 1, wherein the plurality of apertures in said fragrance holder is in substantial alignment with said bores in said door.

6. A fragrance dispensing device comprising:
a vent housing having an upper surface,
an opening in said upper surface through which a fragrance holder is placed within the housing, and
a door hingedly attached to the upper surface over the opening,
wherein the fragrance holder includes a bottom surface having a plurality of apertures capable of passing air therethrough,
wherein the door includes a plurality of bores.

7. The fragrance dispensing device as recited in claim 6, wherein the housing further includes a plurality of air flow vanes in parallel alignment with the opening.

8. The fragrance dispensing device as recited in claim 6, wherein the plurality of apertures in said fragrance holder are in substantial alignment with said plurality of bores in said door.

9. A fragrance dispensing device comprising:
a vent in fluid communication with an air distribution duct,
an opening in said vent to access a fragrance holding surface, and
a door rotatably attached over the opening.

10. The fragrance dispensing device as recited in claim 9, wherein the fragrance holding surface includes a plurality of apertures capable of passing air therethrough.

11. The fragrance dispensing device as recited in claim 9, wherein the door includes a plurality of bores capable of passing air therethrough.
